(19) United States
(12) Patent Application Publication
(10) Pub. No.: US 2016/0095973 A1
(43) Pub. Date: Apr. 7, 2016

(54) Collapsible and Expandable Corrugated Hose with Directional Hand-Pump for Colon Hydrotherapy

(71) Applicant: Delmar Vogel, North Vancouver (CA)

(72) Inventor: Delmar Vogel, North Vancouver (CA)

(21) Appl. No.: 14/505,038

(22) Filed: Oct. 2, 2014

(51) Int. Cl. A61M 3/02

(52) U.S. Cl. CPC A61M 3/0279; A61M 3/0262; A61M 2210/1067

(57) ABSTRACT

A corrugated collapsible and expandable hose specifically for use in colon hydrotherapy which is collapsed for shipping and storage purposes and can be expanded to its full length or part of its full length for use during a colon hydrotherapy treatment and in order to accommodate specific treatment room set-ups. The invention includes a non-corrugated directional hand-pump section which allows the practitioner to apply pumping action to the hose to increase treatment efficacy. This directional hand-pump section is similar to a primer bulb but without valves to restrict direction of flow. Rather, the practitioner may fold the tubing at a crimpable indentation while squeezing the hand pump section to simultaneously stop water flow and control the direction of the pumping action.

### STATEMENT REGARDING FEDERALLY FUNDED RESEARCH

This invention was not made by any agency of the United States Government nor under any contract with any agency of the United States Government.

### TECHNICAL FIELD OF THE INVENTION

The invention relates to medical supplies and more particularly to colon irrigation supplies.

### BACKGROUND OF THE INVENTION

For many years it has been standard for hoses to use a corrugated structure to permit collapsing and expanding of corrugations, thereby reducing storage space and shipping costs, and allowing adjustability to a variety of lengths, as seen in the prior art. This invention is concerned with the application of that corrugation technology to colon hydrotherapy hoses (FIGS. 5 and 6), and to the addition of the directional hand-pump section. (FIGS. 1, 2, 3, & 4 section 2).

The directional hand-pump section (FIGS. 1, 2, 3, & 4 section 2) is similar to a primer bulb pump, of which there are various types as seen in the prior art. This invention improves upon earlier designs by eliminating all inner parts and allowing for a one-piece device, which is simpler and more cost-effective to produce, allowing for the product to be re-usable or disposable for one-time use. Rather than the valves that earlier designs have used to restrict flow to one direction, this invention uses a crimpable indentation (FIGS. 1,2,3, 4, & 7 section 3) which the practitioner may fold to pinch the hose closed, cutting off flow and allowing for pumping force to go in only one direction.

### PRIOR ART

### Corrugated Hoses:

### American Patents:

U.S. Pat. No. 3,908,704 Corrugated Tubing

U.S. Pat. No. 3,409,224 Flexible Drinking Tube

U.S. Pat. No. 5,507,319 Synthetic Resin Bellows Pipe

U.S. Pat. No. 5,704,401 Washing Machine Hose

U.S. Pat. No. 7,677,271 B2 Portable Flexible and Extendable Drain Pipe

U.S. Pat. No. 7,597,119 B2 Flexible and Extendable Hose For Holding Tank Drainage

### Canadian Patents:

(11) CA 2022078 Interconnecting Corrugated Hose

(11) CA 2385291 Cuffed Hose and Method of Manufacture

(11) CA 2145951 Rubber Tubing with Axially Spaced Annularly Corrugated Flexible Segments

### Priming Pumps

### U.S. Patents:

U.S. Pat. No. 2,954,048 Pump and Valve Therefore

U.S. Pat. No. 3,987,775 Squeeze-Tube Primer for Internal Combustion Engines

U.S. Pat. No. 5,970,935 Priming Pump

### BRIEF SUMMARY OF THE INVENTION

A corrugated collapsible and expandable hose specifically for use in colon hydrotherapy which is collapsed for shipping purposes and can be expanded to its full length or part of its full length in order to accommodate specific treatment room set-ups. It includes a non-corrugated directional hand-pump section with crimpable indentations which allow for the cutting off of water flow and the concentration of pumping action in one direction only.

### DETAILED DESCRIPTION OF THE INVENTION

The invention is a one-piece corrugated hose specifically for use in colon hydrotherapy. It can be re-usable or disposable one-time use.

The corrugations extending outward from the tube have side walls of unequal length (FIGS. 1 & 3) and in close-packed condition the side walls are inclined to the axis at an acute angle, producing a contour called cross-cut saw-toothing or oil-canning which will permit one corrugation to fold in under an adjoining corrugation in order to collapse the tube and decrease the total length of said tube (FIGS. 2 & 4), both to reduce packaging size, storage space and shipping costs, and to adjust the hose length for usage in different treatment room layouts by partially or fully extending the corrugation of the tube. The corrugations allow for the hose to be shortened and extended simply by pushing or pulling lengthwise, to any desired length between 1 and 4 feet in the preferred embodiment, although this could be different in an alternate embodiment.

The tubing may also include a non-corrugated section: the directional hand-pump section (FIGS. 1, 2, 3 & 4, section 2). This is similar to a primer bulb pump, but without valves to restrict direction of flow. Rather, the practitioner may fold the tubing at a crimpable indentation (FIGS. 1, 2, 3, 4, & 7, section 3) while squeezing the hand pump section to simultaneously stop water flow and control direction of pumping action. The preferred embodiment includes two crimpable indentations (FIG. 7 section 3) though there may be any number of crimpable indentations. The practitioner may elect to simply squeeze the pump portion of the tube without folding the tube, thereby continuing water flow and allowing the pumping action to radiate in both directions. The pumping action assists in removing waste material from the colon more readily and moving the colon waste material along the tube. The hand pump section can be at any location of the tube and can have various shapes, though in the preferred embodiment it is located closer to one of the hose which means that pumping action can be greater in the tube if the hand pump section is located closer to the colon hydrotherapy equipment or it can be greater in the colon if the hand pump is located closer to the patient. The hand pump can be with or without structural raised ribbing of various sizes and location (FIG. 1, 2, 3, 4, section 4) to increase the strength of the pump walls thus providing increased bounce-back capabilities to its original shape after the bulb is squeezed.

The invention is applicable to hoses of any length and diameter.

### BRIEF DESCRIPTION OF THE DRAWINGS

FIG. 1 shows the Collapsible and Expandable Corrugated Hose with Directional Hand-Pump for Colon Hydrotherapy in extended form, 3D perspective

FIG. 2 shows the Collapsible and Expandable Corrugated Hose with Directional Hand-Pump for Colon Hydrotherapy in collapsed form, 3D perspective

FIG. 3 shows the Collapsible and Expandable Corrugated Hose with Directional Hand-Pump for Colon Hydrotherapy in extended form, 2D perspective

FIG. 4 shows the Collapsible and Expandable Corrugated Hose with Directional Hand-Pump for Colon Hydrotherapy in collapsed form, 2D perspective

FIG. 5 shows the Collapsible and Expandable Corrugated Hose with Directional Hand-Pump for Colon Hydrotherapy in use with the squeeze the pump portion of the tube closer to the colon hydrotherapy device than to the patient allowing for greater pump effect in the tube.

FIG. 6 shows the Collapsible and Expandable Corrugated Hose with Directional Hand-Pump for Colon Hydrotherapy in use with the squeeze the pump portion of the tube closer to the colon hydrotherapy patient than to the device allowing for greater pump effect in the patient's colon.

FIG. 7 shows the Collapsible and Expandable Corrugated Hose with Directional Hand-Pump for Colon Hydrotherapy with the tubing folded at the crimpable indentation causing the hose to flatten and pinch closed to stop water flow and control direction of pumping action.

What is claimed:

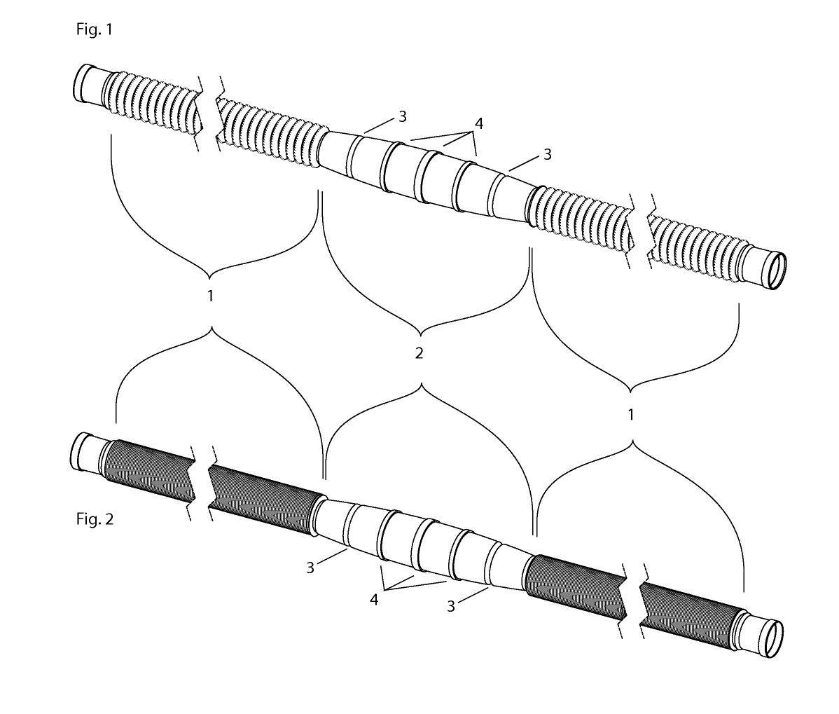

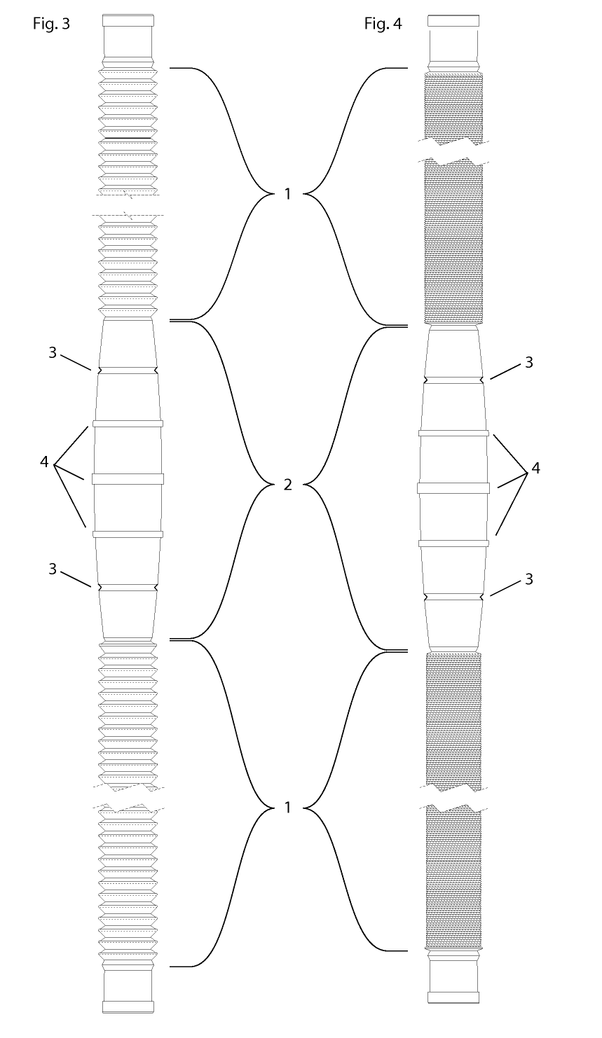

1. A hose, intended for use in colon hydrotherapy, comprising a continuous length of thermoplastic material, most of the length of which is corrugated (FIGS. 1, 2, 3, & 4). The corrugations are annular in the preferred embodiment but may also be helical. The corrugations extending outward from the tube have side walls of unequal length (FIGS. 1 & 3) and in close-packed condition the side walls are inclined to the axis at an acute angle, producing a contour called cross-cut saw-toothing or oil-canning which will permit one corrugation to fold in under an adjoining corrugation in order to collapse the tube (FIGS. 2 & 4) and thereby decrease the total length of said tube and packaging size, which in turn reduces storage space and shipping costs.

2. A corrugated hose, as recited in claim 1, wherein the corrugations (FIGS. 1, 2, 3 & 4) allow for the hose to be shortened and extended simply by pushing or pulling lengthwise, to any desired length between 1 and 4 feet in the preferred embodiment, although this could be different in an alternate embodiment. It is not limited to a specific length or diameter of the tube. This adjustability of length allows for the hose to be configured to different treatment facility layouts. FIGS. 1 and 3 depict the hose in its expanded form, while FIGS. 2 and 4 depict it in its retracted form.

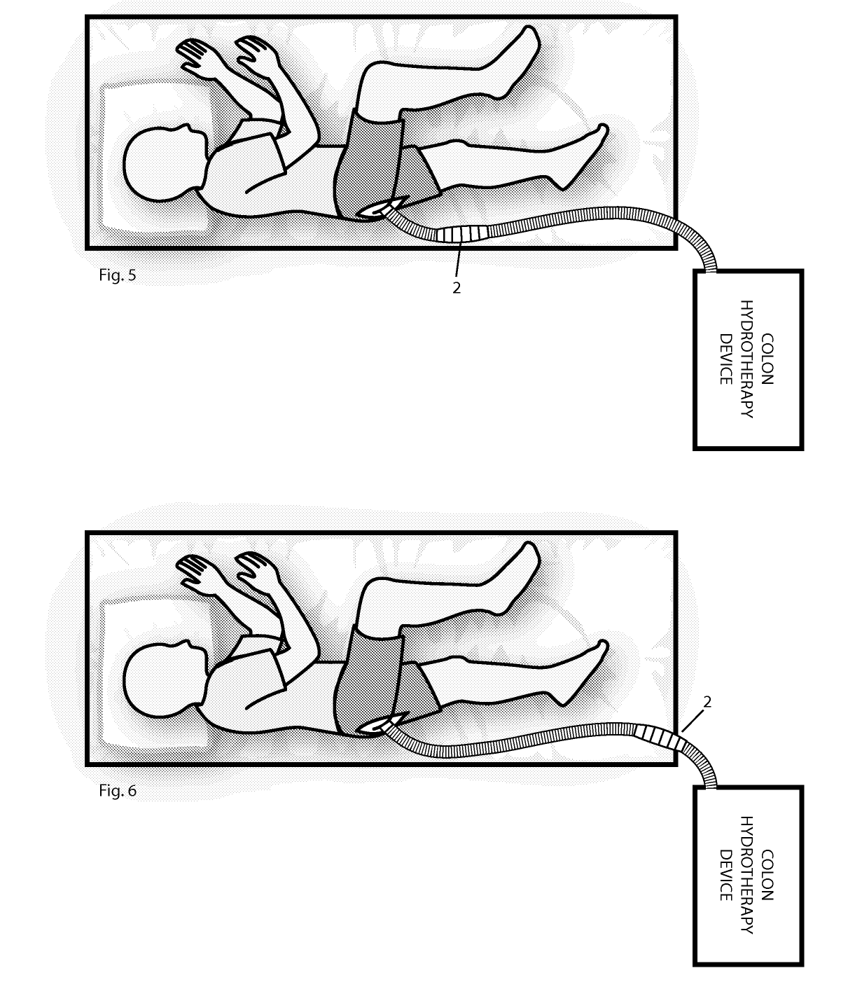

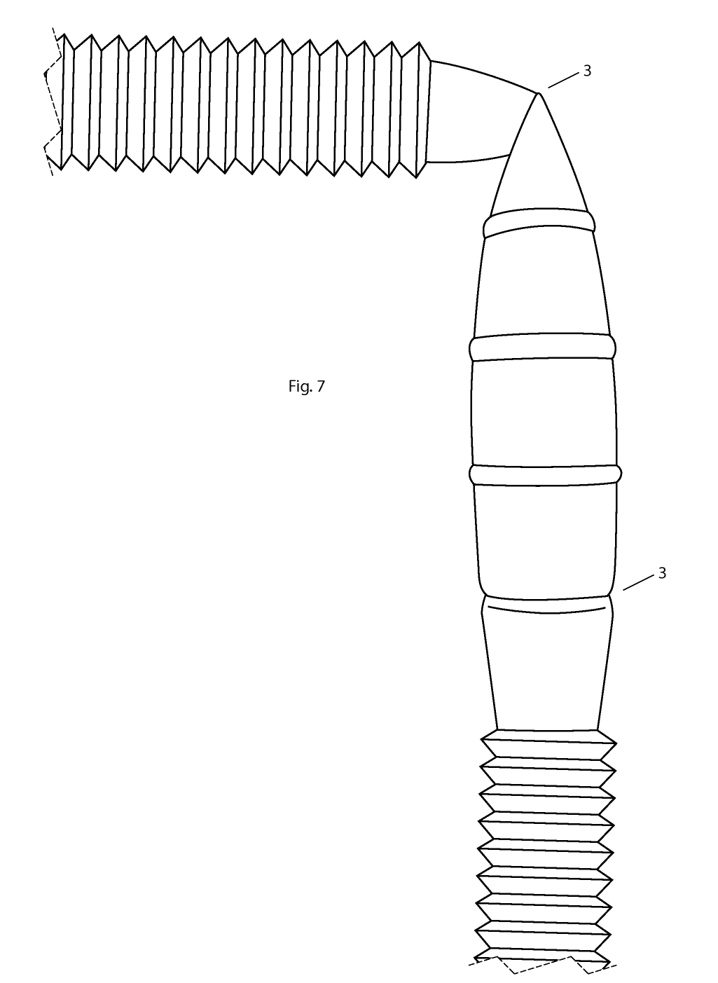

3. A corrugated hose, including a non-corrugated section: the directional hand-pump section (FIGS. 1, 2, 3 & 4, section 2). This is similar to a primer bulb pump, but without valves to restrict direction of flow. This squeezable pump section is, in the preferred embodiment, closer to one end of the hose, and its location is thereby adjustable by placing the hand-pump section of the tube closer to the colon hydrotherapy equipment for greater pump effect in the tube (FIG. 6, section 2) or closer to the anal speculum (FIG. 5, section 2) for greater pump effect in the colon (FIG. 5). In the preferred embodiment, there is only one hand-pump section, although there could be any number. The hand-pump section can be of various shapes and sizes and can be at any location of the tube.

4. A corrugated hose, including a non-corrugated section as recited in claim 3 wherein the practitioner may fold the tubing at a crimpable indentation (FIG. 7, section 3) causing the hose to flatten and pinch closed, while squeezing the hand pump section to simultaneously stop water flow and control direction of pumping action. In the preferred embodiment, there are two crimpable indentations, one at each end of the hand-pump section, although there could be any number, and at any point on the hose.

5. A hand pump that can be with or without structural raised ribbing of various sizes and location (FIGS. 1, 2, 3, & 4, section 4) to increase the strength of the pump walls thus providing increased bounce-back capabilities to its original shape after the bulb is squeezed.
